(19) United States
(12) Patent Application Publication
(10) Pub. No.: US 2007/0249573 A1
(43) Pub. Date: Oct. 25, 2007

(54) 17alpha-substituted 4-(3-oxoestra-4,9-dien-11beta-yl)-benzoic acid, its derivatives and process for its production

(76) Inventors: Gerd Schubert, Jena (DE); Uwe Kaden, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/783,890

(22) Filed: Apr. 12, 2007

Related U.S. Application Data
(60) Provisional application No. 60/791,436, filed on Apr. 13, 2006.

(51) Int. Cl. A61K 31/57; A61K 31/56

(52) U.S. Cl. 514/178; 552/610

(57) ABSTRACT

This invention relates to 17α-substituted 4-(3-oxoestra-4,9-dien-11β-yl)-benzoic acids and derivatives thereof, a process for the production of these compounds, the use of the compounds for the production of pharmaceutical agents as well as pharmaceutical compositions that contain these compounds.

This application claims the benefit of U.S. Ser. No. 60/791,436, filed on Apr. 13, 2006, which is incorporated by reference herein.

This invention relates to 17α-substituted 4-(3-oxoestra-4,9-dien-11β-yl)-benzoic acids and derivatives thereof, a process for the production of these compounds, the use of the compounds for the production of pharmaceutical agents as well as pharmaceutical compositions that contain these compounds.

11β-Aryl-substituted estra-4,9-dienes have been described several times (EP 057 115; G. Teutsch, D. Philibert, Human Reproduction Volume 9 Suppl. 1 1994, 12-31; W. G. E. J. Schoonen, G. J. Vermeulen, G. H. Deckers, P. M. Verbost, H. J. Kloosterboer, Current Topics in Steroid Research 1999, 2, 15-54).

Various 11β-benzaldehyde derivatives of estra-4,9-dien-3-ones are described in, for example, DE 35 04 421, DE 4332283, WO 01/44267, WO 99/45023, DE 102 1034, WO 02/38582 or WO 04/014935. Because of its advantageous progesterone-receptor modulatory action and low antigluocorticoidal action, a representative of this group, the 4-[17β-methoxy-(17α-(methoxymethyl)-3-oxoestra-4,9-dien-11β-yl]benzaldehyde-1(E)-oxime (Asoprisnil) and its derivatives, is of general interest for the treatment of hormone-dependent diseases of women, such as, for example, for the treatment of endometriosis, fibroids or other gynecological dysfunctions, for use in hormone replacement therapy (HRT) as well as for female birth control (WO01/15679A2, WO01/34126A2, and WO01/26603A2).

The object of this invention is to find alternative compounds to those of the prior art, which exhibits a comparable dissociation of action relative to progesterone and glucocorticoid receptors.

The object is achieved, according to the invention, by the preparation of 11β-substituted estra-4,9-dien-3-ones of general formula (I)

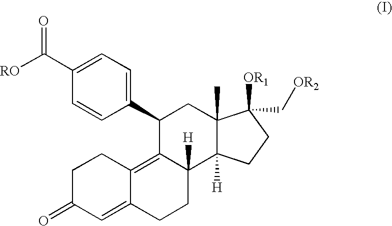

in which

R means a hydrogen atom or an alkyl radical with 1-4 carbon atoms,

R₁ means a hydrogen atom or an alkyl radical with 1-4 carbon atoms,

R₂ means a hydrogen atom or an alkyl radical with 1-4 carbon atoms, as well as the pharmaceutically acceptable salts thereof.

In addition, this invention comprises the new compounds as pharmaceutical active ingredients, the production thereof, their therapeutic application and pharmaceutical dispensing forms that contain the new substances.

The compounds of general formula (I) according to the invention or the pharmaceutically acceptable salts thereof can be used for the production of a pharmaceutical agent, in particular for treatment and prophylaxis of hormone-dependent diseases of women, such as, for example, for the treatment of endometriosis, fibroids or other gynecological dysfunctions, for the use in hormone replacement therapy (HRT) as well as for female birth control.

Moreover, a subject of this invention is also a process for the production of the compounds of general formula (I), in which compounds of general formula (II)

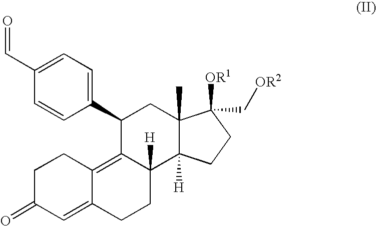

in which
R₁ means a hydrogen atom or an alkyl radical with 1-4 carbon atoms,
R₂ means a hydrogen atom, an alkyl radical with 1-4 carbon atoms, a tetrahydropyranyl radical, a silylalkyl radical with 3-6 carbon atoms or an acyl radical with 1-3 carbon atoms,
   are oxidized as described below to form the corresponding 11β-benzoic acids and then existing protective groups are cleaved off, if necessary. The functional acid group optionally can be esterified. According to the invention, the precursors that correspond to the 11β-benzaldehydes, such as, for example, oximes, hydrazones and similar derivatives or else 11β-formylpentylacetals of estra-4,9-dien-3-ones, are likewise suitable as educts for the process according to the invention.

C₁-C₄-Alkyl groups are defined as saturated or unsaturated, branched or unbranched alkyl radicals. In this case, this is, for example, a methyl, ethyl, n-propyl, iso-propyl, n-, iso- or tert-butyl group.

In terms of R¹, R² and R, methyl or ethyl is preferred.

The silylalkyl groups are, e.g., trimethylsilyl-(TMS) or tert-butyldimethylsilyl ether (TBDMS).

Acyl is preferably derived from C₁-C₃-carboxylic acids. By way of example, formic acid, acetic acid, and propionic acid can be mentioned. Formic acid and acetic acid are preferred.

The compounds that are mentioned below are preferred according to the invention:
1) 4-[17β-Hydroxy-(17α-(methoxymethyl)-3-oxoestra-4,9-dien-11β-yl]-benzoic acid
2) 4-[17β-Methoxy-(17α-(methoxymethyl)-3-oxoestra-4,9-dien-11β-yl]-benzoic acid
3) 4-[17β-Hydroxy-(17α-(hydroxymethyl)-3-oxoestra-4,9-dien-11β-yl]-benzoic acid
4) 4-[17α-(Acetoxymethyl)-17β-hydroxy 3-oxoestra-4,9-dien-11β-yl]-benzoic acid
5) 4-[17β-Methoxy-(17α-(methoxymethyl)-3-oxoestra-4,9-dien-11β-yl]-benzoic acid methyl ester
6) 4-[17β-Methoxy-(17α-(methoxymethyl)-3-oxoestra-4,9-dien-11β-yl]-benzoic acid ethyl ester

For the production of the compounds of general formula (I) according to the invention, 11β-benzaldehydes of 17α-substituted CH₂OR₂-estra-4,9-dien-3-ones of formula II

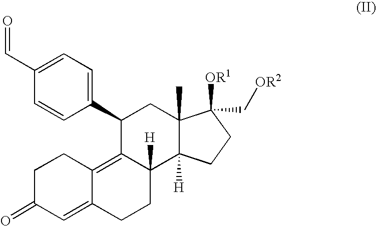

in which
R₁ means a hydrogen atom or an alkyl radical with 1-4 carbon atoms,
R₂ means a hydrogen atom, an alkyl radical with 1-4 carbon atoms, a tetrahydropyranyl radical, a silylalkyl radical with 3-6 carbon atoms, or an acyl radical with 1-3 carbon atoms,
whereby the derivatives of the corresponding 11β-benzaldehydes, such as, for example, oximes, hydrazones and similar derivatives, or else 11 formylphenylacetals of estra-4,9-dien-3-ones, are first intermediately reacted to form the corresponding 11β-benzaldehydes of general formula (II),
   are oxidized under acidic conditions, such as chromic acid in acetone (Jones oxidation) or under neutral conditions, such as pyridinium chlorochromate in dimethylformamide, or under alkaline conditions, such as silver nitrate in aqueous NaOH or hexafluoroacetone and H₂O₂ in the presence of Na₂CO₃ or tetrabutylammonium permanganate in pyridine to form the corresponding 11β-benzoic acid.

Then, still present protective groups of the alcoholic groups of the D ring are hydrolyzed under acidic or alkaline conditions and/or the 11β-benzoic acid is esterified, if necessary.

The 17α-substituted 4-(3-oxoestra-4,9-dien-11β-yl)benzoic acids of general formula I according to the invention are produced as described below:

3,3-Dimethoxy-estra-4,9-dien-17-one (III) [Pierdet, A.; Vignau, M.; French Pat. 5183 (1966)] is converted into 3,3-dimethoxy-5α,10α-epoxy-estr-9(11)-en-17-one (IV) in a way that is known in the art with H₂O₂ in the presence of hexafluoroacetone and sodium bicarbonate in methylene chloride and pyridine [Costerousse, G.; Teutsch, G. EP 5100 (1979); Teutsch, G.; Ojasoo, T.; Raynaud, J. P., J. Steroid Biochem. (1988), 31, 549 to 565].

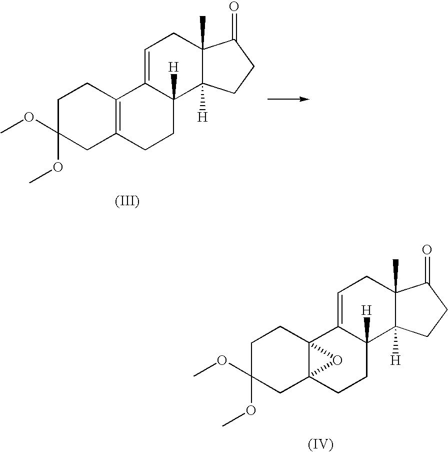

While (V) is obtained, an 11β-(4-dimethylacetal-benzaldehyde) grouping is introduced by a Cu(I)-salt-catalyzed Grignard reaction with a 4-bromobenzaldehyde acetal. [DE 4332283 as well as Teutsch, G., Bélanger, Tetrahedron Letters (1979), 22, 2051-2054].

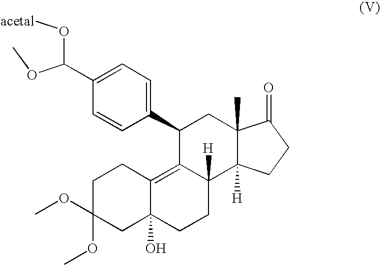

According to Corey and Chaykowsky [J. Amer. Chem. Soc (1962), 84, 3782], the 17-carbonyl group is converted with trimethylsulfonium iodide in dimethylformamide or dimethyl sulfoxide in the presence of potassium tert-butanolate into spiroepoxide (VI).

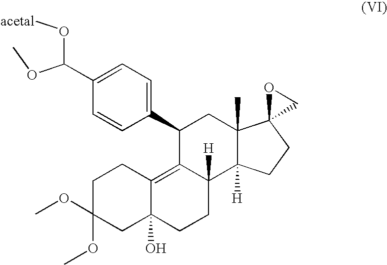

The ring opening with sodium methylate in MeOH yields a 17α-(methoxymethyl)-17β-hydroxy compound (VIIa). By acidic hydrolysis of this compound with dilute acetic acid or p-toluenesulfonic acid in acetone, the 11β-benzaldehyde (IIa) (DE 4332283) is formed with simultaneous cleavage of the protective groups.

After the 17β-hydroxyl group of (VIIa) is etherified with methyl iodide and potassium tert-butanolate, (VIIb) is produced. Subsequent acidic hydrolysis of (VIIb) results in 11β-aldehyde (IIb) (DE 4332283).

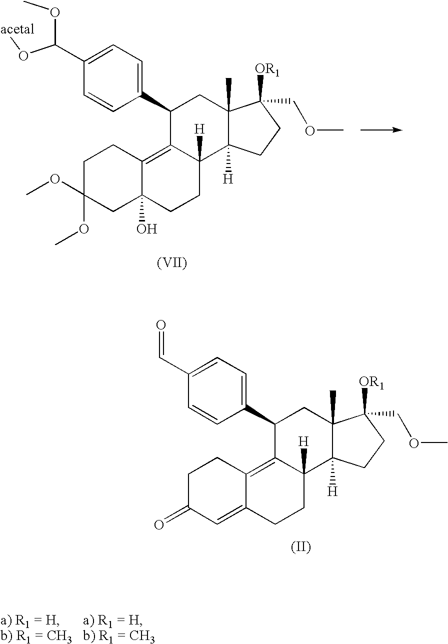

As an alternative to this, the ring opening of the spiroepoxide (VI) with aqueous NaOH in N-methyl-2-pyrrolidone yields a 17β-hydroxy-17α-(hydroxymethyl) compound (VIII).

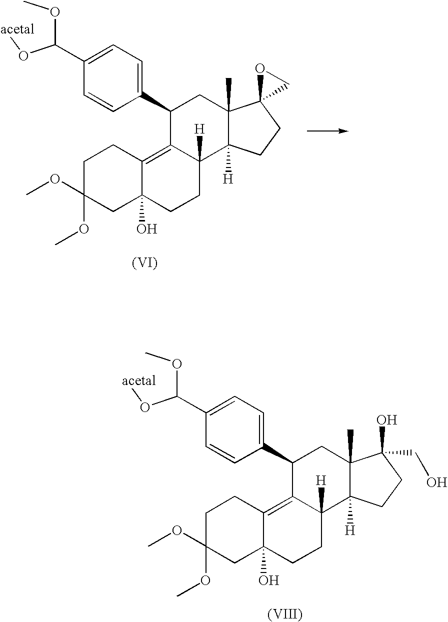

The 11β-benzaldehyde (IIc) (WO 04/014935) is produced by treatment with acids.

A 17α-acetoxymethyl compound (IId) can be formed selectively with acetic anhydride in the presence of pyridine. The primary hydroxyl group can also be protected as tetrahydropyranyl ether (IIe) or as silylether.

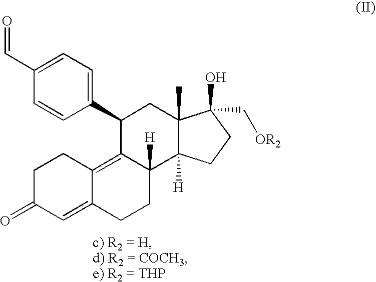

The oxidation of the 11β-benzaldehyde grouping to form 11β-benzoic acid is carried out in the 17α-substituted compounds (IIa), (IIb) and (IId) with Jones reagent (chromium trioxide in sulfuric acid and acetone) or in (IIa), also with silver nitrate and lye. For the selective oxidation of the 11β-benzaldehyde group, a protection of the primary hydroxyl group is necessary in the case of the 17α-CH₂OH compound (IIc) to avoid an oxidation of this grouping. As a protection of the primary hydroxyl group, the acetate (IId) or else a 17α-CH₂O-tetrahydropyranyl ether derivative (IIe) is suitable.

(IIe) is oxidized to 11β-benzoic acid under neutral or alkaline conditions, for example with silver nitrate and lye. Then, the protective group (17α-CH₂OTHP) is removed again under acidic conditions, and (Ic) is obtained.

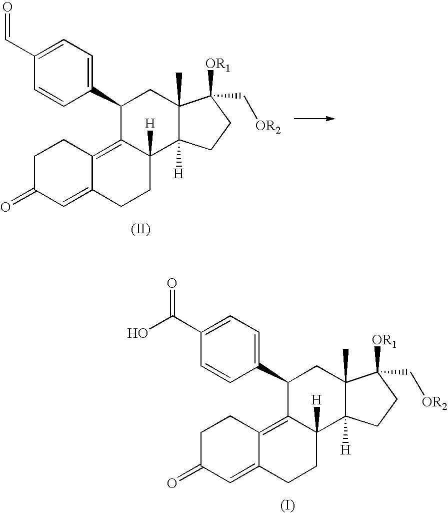

By reaction of the 11β-benzoic acid (Ib) with thionyl chloride in pyridine, the acid chloride, which can be converted with alcohols directly into the esters (Ie) or (If), can be formed.

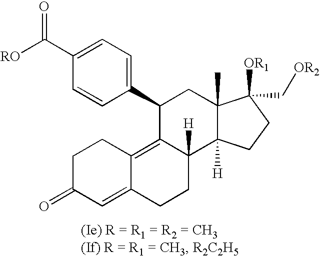

The examples below are used for a more detailed explanation of the subject of the invention, without being limited to the latter.

### EXAMPLE 1

### 4-[17β-Hydroxy-17α-(methoxymethyl)-3-oxoestra-4,9-dien-11β-yl]-benzoic Acid (Ia)

Method A

420 mg of 4-[17β-hydroxy-(17α-(methoxymethyl)-3-oxoestra-4,9-dien-11β-yl]benzaldehyde (IIa) is dissolved in 30 ml of acetone. At 0 to 5° C., JONES reagent is added in drops until an orange-colored solution can be detected. After 1 ml of isopropanol is added, it is diluted with ice water, and the dark green solution is extracted with methylene chloride.

The solution is washed neutral, dried, and concentrated by evaporation. After purification by preparative layer chromatography on silica gel, the crude product is recrystallized from acetone. 155 mg of 4-[17β-hydroxy-(17α-(methoxymethyl)-3-oxoestra-4,9-dien-11β-yl]-benzoic acid (Ia) is obtained.

Method B

While being stirred in a solution that consists of 1.2 g of sodium hydroxide in 70 ml of water, 2.55 g of silver nitrate in 70 ml of water is added in drops. 2.1 g of 4-[17β-hydroxy-17α-(methoxymethyl)-3-oxoestra-4,9-dien-11β-yl]benzaldehyde (IIa) (dissolved in 40 ml of tetrahydrofuran) is added to this suspension within 20 minutes and stirred for another 1.5 hours at 60-65° C. 12 ml of 2N hydrochloric acid is added in drops to the cooled reaction solution, then it is extracted with chloroform. The solution is washed, dried, and largely concentrated by evaporation in a vacuum. It can be crystallized from methanol, and 1.17 g of 4-[17β-hydroxy-(17α-(methoxymethyl)-3-oxoestra-4,9-dien-11β-yl]-benzoic acid (Ia) is obtained.

Melting point: 170 to 176° C. (acetone)

α_{D}=+174° (CHCl₃);

¹H-NMR (CDCl₃): 7.94 (d, 2H, J=8.4, H-3′), 7.26 (d, 2H, J=8.4, H-2′), 5.78 (s, 1H, H-4), 4.43 (d, 1H, J=7.2, H-11), 3.55 (d, 2H, J=9.6, CH₂O), 3.41 (s, 3H, OCH₃), 3.23 (d, 2H, J=9.2, CH₂O), 0.51 (s, 3H, H-18).

¹³C-NMR (CDCl₃): 200.0, 168.5, 157.3, 150.1, 145.5, 130.0, 129.6, 127.8, 126.8, 122.8, 82.5, 59.0, 50.7, 49.6, 48.7, 45.9, 40.9, 39.1, 38.6, 36.6, 33.7, 31.1, 30.9, 27.5, 25.7, 23.6, 15.6.

### EXAMPLE 2

### 4-[17β-Methoxy-17α-(methoxymethyl)-3-oxoestra-4,9-dien-11β-yl]-benzoic Acid (Ib)

2 g of 4-[17β-methoxy-(17α-(methoxymethyl)-3-oxoestra-4,9-dien-11β-yl]benzaldehyde (IIb) is dissolved in 500 ml of DMF. At 0 to 5° C., JONES reagent is added in drops until an orange-colored solution is left. After 3 ml of isopropanol is added, it is diluted with ice water, and the solution is extracted with methylene chloride. Then, the organic phase is washed neutral, dried and concentrated by evaporation and recrystallized from acetone. 1.07 g of 4-[17β-methoxy-(17α-(methoxymethyl)-3-oxoestra-4,9-dien-11β-yl]-benzoic acid (Ib) is obtained.

Melting point 163 to 181° C. (MeOH)

α_{D}=+162° (CHCl₃);

¹H-NMR (CDCl3): 8.00 (d, 2H, J=8.4), 7.31 (d, 2H, J=8.4), 5.80 (s, 1H, H-4), 4.43 (d, 1H, J=7.2, H-11), 3.57 (d, 2H, J=9.6, CH₂O), 3.41 (s, 3H, OCH₃), 3.40 (d, 2H, J=9.2, CH₂O), 3.26 (s, 3H, OCH₃), 0.51 (s, 3H, H-18).

### EXAMPLE 3

### 4-[17β-Hydroxy-17α-(hydroxymethyl)-3-oxoestra-4,9-dien-11β-yl]-benzoic Acid (Ic)

160 mg of 4-[17β-hydroxy-17α-(tetrahydropyranyloxy)methyl-3-oxoestra-4,9-dien-11β-yl]-benzoic acid is stirred for 5 hours at 60° C. in 30 ml of 80% acetic acid. After extraction with chloroform, washing and drying, 90 mg of resin is obtained by concentration by evaporation in a vacuum, which is purified with preparative layer chromatography on silica gel with the mobile solvents toluene/ethanol 2:1 and toluene/acetone 2:1. 4-[17β-Hydroxy-17α-(hydroxymethyl)-3-oxoestra-4,9-dien-11β-yl]-benzoic acid (Ic) is recrystallized from methanol.

Melting point 207 to 212° C. (MeOH)

¹H-NMR (CDCl3): 7.94 (d, J=8.4, H-3′), 7.26 (d, J=8.4, H-2′), 5.78 (s, H-4), 4.42 (d, J=7.1, H-11), 3.73 (d, J=11.3, CH₂O), 3.41 (d, J=11.3, CH₂O), 2.71 (dt, J=15.1, 4.8, H-1), 2.06 (m, OH) 0.52 (s, H-18).

¹³C-NMR (CDCl3): 200.0, 168.6, 157.2, 150.1, 145.3, 130.0, 129.6, 127.9, 126.8, 122.8, 83.3, 66.3, 50.6, 45.7, 40.8, 39.2, 38.3, 36.6, 33.0, 31.1, 27.5, 25.7, 23.6, 15.6.

Production of the Starting Compound:

Stage I

19.4 g of 4-(3,3-dimethoxy-5α-hydroxy-17-(S)-spiroepoxy-estr-9-en-11β-yl)benzaldehyde-ethylene acetal is dissolved in 250 ml of N-methyl-2-pyrrolidone. 145 ml of 2N aqueous NaOH is added in drops, it is heated for 2 hours to 100° C., cooled off, and added in drops in 250 ml of aqueous 10% NH₄Cl solution. After extraction with ethyl acetate, the organic phase is washed neutral, dried, and concentrated by evaporation under vacuum. 19.5 g (yield 65%) of 4-[3,3-dimethoxy-5α, 17β-dihydroxy-17α-(hydroxymethyl)-estr-9-en-11β-yl]-benzaldehyde-ethylene acetal is obtained as a crude product.

¹H-NMR (CDCl3): 7.37 (d, 2H, J=8.0, H-3′), 7.23 (d, 2H, J=8.0, H-2′), 5.76 (s, 1H, benzaldehyde acetal), 4.42 (d, 1H, J=7.2, H-11), 4.07 (m, 4H, ethylene acetal), 3.74 (d, 2H, J=10.8, CH₂O), 3.40 (d, 2H, J=10.8, CH₂O), 3.21 and 3.22 (2s; 3H in each case, 2×OCH₃), 0.47 (s, 3H, H-18).

The crude product is dissolved in 120 ml of tetrahydrofuran, mixed with 12 ml of water and 5.3 g of p-toluenesulfonic acid, and it is stirred for 4 hours at room temperature. Then, the solution is neutralized with aqueous NaHCO₃ solution, and after the usual working-up process, 8.5 g of 4-[17β-hydroxy-17α-(hydroxymethyl)-3-oxoestra-4,9-dien-11β-yl]-benzaldehyde is isolated with ethyl acetate as a light yellow foam, which is purified by flash chromatography (yield 65%).

Melting point: 116 to 123° C. (acetone)

α_{D}=+185° (CHCl₃)

¹H-NMR: 9.98 (CHO), 7.00 (d, 2H, J=8.0), 6.67 (d, 2H, J=8.0), 5.74 (s, 1H, H-4), 4.32 (d, 1H, J=7.4, H-11), 3.79 (d, 2H, J=10.8, CH₂O), 3.43 (d, 2H, J=10.8, CH₂O), 0.60 (s, 3H, H-18).

Stage 2

5.4 g of 4-[17β-hydroxy-17α-(hydroxymethyl)-3-oxoestra-4,9-dien-11β-yl]benzaldehyde is reacted in 50 ml of methylene chloride with 16.9 ml of 3,4-dihydro-2H-pyran and 335 mg of pyridinium-4-toluenesulfonate within 1 hour at room temperature. Then, the solution is stirred into 100 ml of saturated aqueous NaHCO₃ solution, extracted with methylene chloride, the organic solution is washed neutral, dried on Na₂SO₄, filtered off, and the solvent is concentrated by evaporation under vacuum. 7.3 g of a mixture that consists of 4-[17β-hydroxy-17α-tetrahydroxypyranyloxy)methyl-3-oxoestra-4,9-dien-11β-yl]benzaldehyde and 4-[17β-tetrahydropyranyloxy, 17α-(tetrahydroxy-pyranyloxy)methyl-3-oxoestra-4,9-dien-11β-yl]benzaldehyde is obtained. After flash chromatography on silica gel with a toluene/acetone gradient, 4.7 g of 4-[17β-hydroxy-17α-(tetrahydropyranyloxy)methyl-3-oxoestra-4,9-dien-11β-yl]-benzaldehyde is isolated.

¹H-NMR: 9.95 (CHO), 7.79 (d, 2H, J=8.0), 7.36 (d, 2H, J=8.0), 5.79 (s, 1H, H-4), 4.56 (2t, 2H, CH₂O), 4.60 (d, 2H, J=10.8, CH₂O), 4.44 (d, 1H, J=6.4, H-11), 0.53 (s, 3H, H-18).

Stage 3

A solution that consists of 240 mg of sodium hydroxide in 14 ml of water is added in drops to 500 mg of silver nitrate in 14 ml of water while being stirred. 475 mg of 4-[17β-hydroxy-17α-(tetrahydropyranyloxy)methyl-3-oxoestra-4,9-dien-11β-yl]-benzaldehyde (IIe) in 10 ml THF is added in drops to this suspension and stirred for another 3 hours at 50-55° C. It is cooled off, 2 ml of 2N hydrochloric acid is added, and it is extracted with chloroform. The solution is washed, dried, and concentrated by evaporation in a vacuum. After flash chromatography on silica gel/toluene/acetone (gradient 4:1 to 1:1), 160 mg of 4-[17β-hydroxy-17α-(tetrahydropyranyloxy)methyl-3-oxoestra-4,9-dien-11β-yl]-benzoic acid is obtained.

### EXAMPLE 4

### 4-[17α-(Acetoxymethyl)-17β-hydroxy-3-oxoestra-4,9-dien-11β-yl]-benzoic Acid (Id)

The production is carried out analogously to Example 1 from 100 mg of 4-[17α-(acetoxymethyl)-17β-hydroxy-3-oxoestra-4,9-dien-11β-yl]-benzaldehyde (IId) with Jones Reagent.

Yield: 33 mg of colorless foam

¹H-NMR: 8.00 (d, J=8.0, H-3′), 7.29 (d, J=8.0, H-2′), 5.80 (s, H-4), 4.44 (d, J=6.8, H-11), 4.21 and 4.08 (2d, 2H, J, 7.2 Hz, CH₂OAc), 2.16 (s, 3H, COCH₃), 0.55 (s, H-18).

Production of the Starting Compound

Stage 1

1 g of the crude product of 4-[3,3-dimethoxy-5α,17β-dihydroxy-17α-(hydroxymethyl)-estr-9-en-11β-yl]-benzaldehyde-ethylene acetal (Example 2, Stage 1) is dissolved in 10 ml of pyridine, mixed with 4 ml of acetic anhydride and stirred for 3 hours at room temperature. It is poured into ice water and extracted with ethyl acetate, washed with water, and the organic phase is concentrated by evaporation after drying under vacuum. The crude product (820 mg) is purified by preparative layer chromatography on silica gel PF_{254+366 nm} with the flow agent toluene/acetone 4:1. 640 mg of 4-[17α-(acetoxymethyl)-3,3-dimethoxy-5α,17β-dihydroxy 11βestr-9-en-11β-yl]benzaldehyde ethylene acetal is obtained as colorless foam.

Stage 2

600 mg of 4-[17α-(acetoxymethyl)-3,3-dimethoxy-5α,17β-dihydroxy-estr-9-en-11β-yl]benzaldehyde ethylene acetal is dissolved in 30 ml of acetone. 0.5 ml of water and 300 mg of p-toluenesulfonic acid are added, stirred for 2 hours at room temperature, and neutralized with aqueous NH₄OH. The product that precipitates in this case is suctioned off and recrystallized from ethyl acetate and then from acetone. 432 mg of 4-[17α-(acetoxymethyl)-17β-hydroxy-3-oxoestra-4,9-dien-11β-yl]-benzaldehyde is obtained.

Melting point: 213 to 220° C. while decomposing (acetone)

α_{D}=+170° (CHCl₃)

¹H-NMR (DMSO): 9.98 (s, 1H, CHO), 7.82 (d, 2H, J=8.4), 7.37 (d, 2H, J=8.4), 5.81 (s, 1H, H-4), 4.47 (d, 1H, J=6.9, H-11), 4.21 (d, 2H, J=11.8, CH₂O), 4.08 (d, 2H, J=11.8, CH₂O), 2.16 (s, 3H, COCH₃), 0.55 (s, 3H, H-18).

### EXAMPLE 5

### 4-[17β-Methoxy-(17β-(methoxymethyl)-3-oxoestra-4,9-dien-11β-yl]benzoic Acid Methyl Ester (Ie)

334 mg of 4-[17β-methoxy-(17α-(methoxymethyl)-3-oxoestra-4,9-dien-11β-yl]benzoic acid is dissolved in 10 ml of THF and 1 ml of pyridine. 0.3 ml of thionyl chloride in 1.5 ml of THF is slowly added in drops. After 30 minutes, methanol is added until a clear solution is produced. It is mixed with water. It is extracted with methylene chloride, the organic phase is washed neutral, dried and concentrated by evaporation. The crude product is purified by preparative layer chromatography on silica gel and recrystallized from acetone. 218 mg of (Ie) is obtained.

Melting point 102-109° C. (acetone)

α_{D}=+160° (CHCl₃);

¹H-NMR: 7.93 (d, 2H, J=8.4 Hz), 7.27 (d, 2H, J=7.8 Hz), 5.78 (s, 1H, H-4), 4.42 (d, 1H, J=6.9, H-11), 3.90 (s, 3H, COOCH₃), 3.57 (d, 2H, J=10.8 Hz, CH₂O), 3.41 (s, 3H, OCH₃), 3.25 (s, 3H, OCH₃), 3.40 (d, 2H, J=10.5, CH₂O), 0.50 (s, 3H, H-18).

1. 17α-Substituted 4-(3-oxoestra-4,9-dien-11β-yl)-benzoic acid of general formula (I) 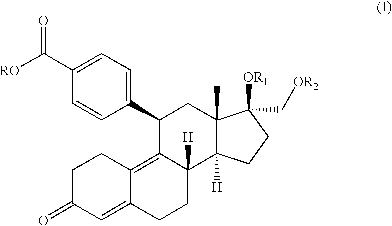
in which
R means a hydrogen atom or an alkyl radical with 1-4 carbon atoms,
R₁ means a hydrogen atom or an alkyl radical with 1-4 carbon atoms,
R₂ means a hydrogen atom or an alkyl radical with 1-4 carbon atoms, as well as the pharmaceutically acceptable salts thereof.

2. 17α-Substituted 4-(3-oxoestra-4,9-dien-11β-yl)-benzoic acid according to claim 1, namely
1) 4-[17β-Hydroxy-(17α-(methoxymethyl)-3-oxoestra-4,9-dien-11β-yl]-benzoic acid
2) 4-[17β-Methoxy-(17α-(methoxymethyl)-3-oxoestra-4,9-dien-11β-yl]-benzoic acid
3) 4-[17β-Hydroxy-(17α-(hydroxymethyl)-3-oxoestra-4,9-dien-11β-yl]-benzoic acid
4) 4-[17α-(Acetoxymethyl)-17β-hydroxy 3-oxoestra-4,9-dien-11β-yl]-benzoic acid
5) 4-[17β-Methoxy-(17α-(methoxymethyl)-3-oxoestra-4,9-dien-11β-yl]-benzoic acid methyl ester
6) 4-[17β-Methoxy-(17α-(methoxymethyl)-3-oxoestra-4,9-dien-11β-yl]-benzoic acid ethyl ester

3. Process for the production of 17α-substituted 4-(3-oxoestra-4,9-dien-11β-yl)-benzoic acid of general formula (I), characterized in that 11β-substituted estra-4,9-dien-3-ones of general formula (II) 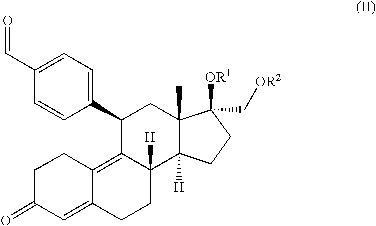
in which
R₁ means a hydrogen atom or an alkyl radical with 1-4 carbon atoms,
R₂ means a hydrogen atom, an alkyl radical with 1-4 carbon atoms, a
tetrahydropyranyl radical, a silylalkyl radical with 3-6 carbon atoms or an acyl radical with 1-3 carbon atoms,
are oxidized under acidic, neutral or alkaline conditions to form the corresponding 11β-benzoic acid.

4. Process according to claim 3, wherein corresponding 11-substituted derivatives of estra-4,9-dien-3-ones are reacted to form compounds of general formula (II).

5. Process according to claim 4, wherein 11-benzaldehyde oximes, 11-benzaldehyde hydrazones or 11β-formylphenyl acetals of estra-4,9-dien-3-ones are reacted to form compounds of general formula (II).

6. Process according to claim 3, wherein the oxidation is performed to form corresponding 11β-benzoic acid under acidic conditions.

7. Process according to claim 6, wherein the acidic oxidation is performed to form corresponding 11β-benzoic acid with chromium trioxide in sulfuric acid and acetone (Jones oxidation).

8. Process according to claim 3, wherein the oxidation is performed to form corresponding 11β-benzoic acid under neutral conditions.

9. Process according to claim 8, wherein the oxidation is performed to form the corresponding 11β-benzoic acid under neutral conditions with pyridinium chlorochromate in dimethylformamide.

10. Process according to claim 3, wherein the oxidation is performed to form the corresponding 11β-benzoic acid under alkaline conditions.

11. Process according to claim 10, wherein the oxidation is performed to form the corresponding 11β-benzoic acid under alkaline conditions with silver nitrate in aqueous NaOH or with hexafluoroacetone and H₂O₂ in the presence of Na₂CO₃ or with tetrabutylammonium permanganate in pyridine.

12. Process according to claim 1, wherein optionally still present protective groups on the D ring are hydrolyzed under acidic or alkaline conditions to form R¹ and/or R².

13. 17α-Substituted 4-(3-oxoestra-4,9-dien-11β-yl)-benzoic acid according to claim 1 for use as a pharmaceutical agent.

14. Pharmaceutical compositions that contain at least one compound according to claim 1.

15. Use of the compounds according to claim 1 for the production of a pharmaceutical agent for treatment and prophylaxis of gynecological diseases such as endometriosis, leiomyomas of the uterus, dysfunctional bleeding and dysmenorrhea.

16. Use of the compounds according to claim 1 for female birth control.

17. Use of the compounds according to claim 1 for the production of a pharmaceutical agent for female hormone replacement therapy.
